(19) United States
(12) Patent Application Publication
(10) Pub. No.: US 2011/0060233 A1
(43) Pub. Date: Mar. 10, 2011

(54) METHODS AND SYSTEM FOR IMPLEMENTING A CLINICAL TRIAL

(76) Inventors: Randol R. Spaulding, West Bend, WI (US); Brock Heinz, Grafton, WI (US); Paul Braemer Schultz, Grafton, WI (US)

(21) Appl. No.: 12/554,200

(22) Filed: Sep. 4, 2009

(51) Int. Cl. A61B 5/04; G06Q 50/00

(52) U.S. Cl. 600/509; 705/3

(57) ABSTRACT

A system and method for automating the implementation of and collection of data during a clinical trial on living subjects includes a computer-based clinical trial management system, programmed to define clinical trial protocols and to store subject data, including a subject identifier; an electronic subject monitoring system, for acquiring physiological data from the subject during the clinical trial; and an electronic subject polling device. The electronic polling device is programmed to acquire physiological data from the monitoring system in accordance with the clinical trial protocols, automatically, electronically associate the physiological data with an appropriate subject ID and transmit the physiological data to the clinical trial management system for storage.

### BACKGROUND

The present invention relates to methods and systems for implementing a clinical trial on a living subject, and particularly to the electronic collection and management of workflow and data during a clinical trial.

### SUMMARY

In one embodiment, the invention provides a system for automating the implementation of and collection of data during a clinical trial on living subjects. The system comprises a computer-based clinical trial management system, programmed to define clinical trial protocols and to store subject data, including a subject identifier; an electronic subject monitoring system, for acquiring physiological data from the subject during the clinical trial; and an electronic subject polling device programmed to acquire physiological data from the monitoring system in accordance with the clinical trial protocols, automatically, electronically associate the physiological data with an appropriate subject ID and transmit the physiological data to the clinical trial management system for storage.

In another embodiment, the invention provides a method of implementing and collecting physiological data during a clinical trial. The method comprises developing an electronic database of potential clinical trial subjects; programming a clinical trial management system with a clinical trial protocol; automatically, electronically selecting the subjects for the clinical trial from the database based on a comparison of the protocol with the database, and associating each selected subject with an identifier; programming a subject monitoring system with the subject identifier and connecting the selected subjects to the subject monitoring system; acquiring ECG data from the subject monitoring system; and transmitting the ECG data to the clinical trial management system in accordance with the protocol.

Other aspects of the invention will become apparent by consideration of the detailed description and accompanying drawings.

### BRIEF DESCRIPTION OF THE DRAWINGS

FIG. 1 is a schematic diagram of the system embodying the invention.

FIG. 2 is a portion of the schematic shown in FIG. 1, illustrating some details of how the test subjects are connected to the subject monitoring system.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limited. The use of “including,” “comprising” or “having” and variations thereof herein is meant to encompass the items listed thereafter and equivalents thereof as well as additional items. The terms “mounted,” “connected” and “coupled” are used broadly and encompass both direct and indirect mounting, connecting and coupling. Further, “connected” and “coupled” are not restricted to physical or mechanical connections or couplings, and can include electrical connections or couplings, whether direct or indirect. Also, electronic communications and notifications may be performed using any known means including direct connections, wireless connections, etc.

It should be noted that a plurality of hardware and software based devices, as well as a plurality of different structural components may be utilized to implement the invention. Furthermore, and as described in subsequent paragraphs, the specific configurations illustrated in the drawings are intended to exemplify embodiments of the invention and that other alternative configurations are possible.

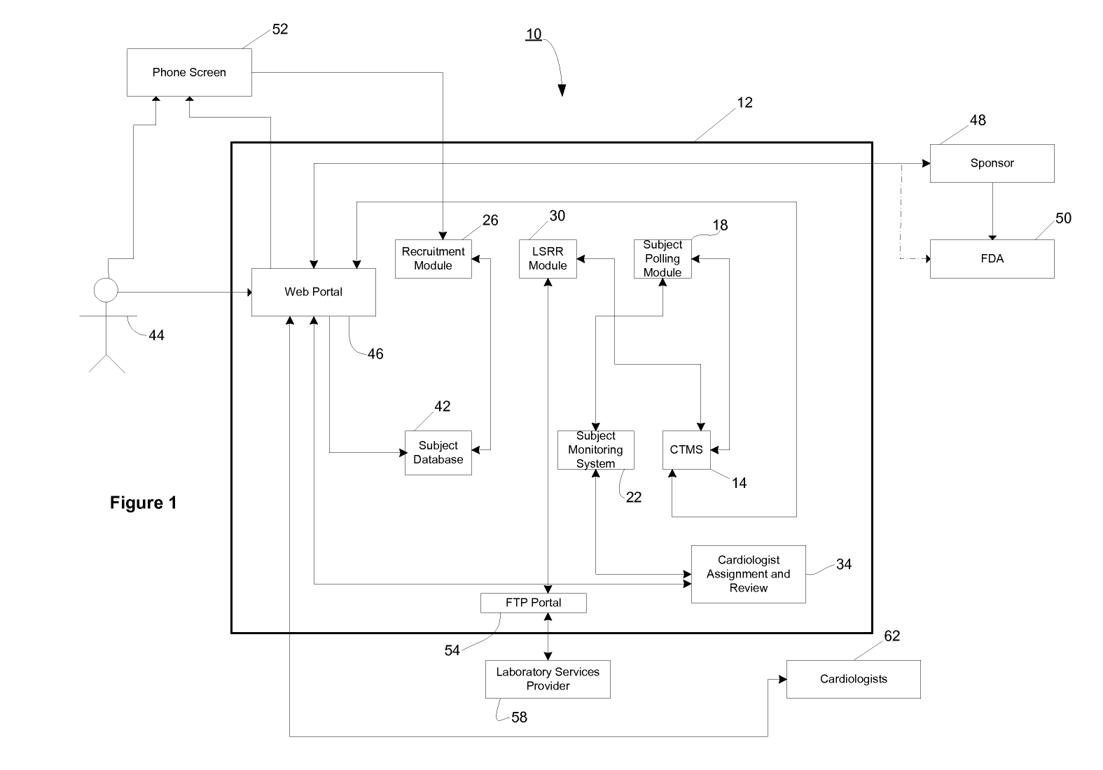

Shown in FIG. 1 is a schematic diagram of a system 10 that automates the implementation of and collection of data during clinical trials performed by a clinical trial service provider 12, also sometimes referred to as a contract research organization (“CRO”). A commercially available embodiment of the system 10 is the integrated system of Electronic Data Capture (“EDC”) applications developed and marketed by Spaulding Clinical Research LLC of West Bend, Wis. In general terms, the system 10 includes multiple applications 14, 18, 22, 26, 30, 34 and 38, in communication with one another over a network, and a subject database 42. The applications include a Clinical Trial Management System (CTMS) 14, a Subject Polling Module (SPM) 18, a Subject Monitoring System (SMS) 22, a Recruitment Module (RM) 26, a Laboratory Scheduling and Receipt of Results (LSRR) module 30, a Cardiologist Assignment and Review (CAR) module 34, and a Video Camera Network (VCN) 38. Each of these systems or modules can run on separate servers or be combined in any combination to run on one or more servers as appropriate. The network can be the a wide or local area network, a virtual private network, an internetwork (such as the internet, an extranet or an intranet) or any combination of these or other networks that allow for communication between hardware and software devices and modules. In addition, the communication can occur through dedicated wired lines, or through wireless communication networks or devices.

The system 10 includes a web portal 46 for facilitating communication between the system 10 and external devices through the internet. Data exported from the system 10 is preferably in a format that allows a near real-time view of the data collected in the clinical trial, and the system 10 supports web-based tools for analyzing and querying for desired data. More specifically, in one embodiment, data sets can be exported in study data tabulation model (“SDTM”) format, and can be imported into statistical analysis system (“SAS”) programming environments with minimal effort. This functionality reduces the time that is traditionally associated with data exported from a CRO. The subject database 42 includes information for subjects that either may participate or do participate in a clinical trial implemented using the system 10.

The CTMS 14 is a software program and associated database for storing data relating to subjects 44 of a clinical trial. A commercially available example of such a program is AlphaDAS, a software-based CTMS developed and marketed by Logos Technologies. A protocol for a clinical trial is manually entered into the CTMS 14. The protocol defines the clinical trial, i.e., the profile of the subjects desired to participate, the medications to be given, the medical parameters of the subjects to be monitored, the diet of the subjects, the duration of the clinical trial and when medication is given, when food is given, and when medical parameters are measured from the subjects, etc. The profile also defines inclusion and exclusion criteria for the test subjects that will participate in the clinical trial. For example, the clinical trial may require only men, or only women, or people of a certain health profile, etc. The CTMS 14 also creates a record for each test subject 44 and automatically assigns an identifier (“ID”) to each subject 44. The subject IDs are confidential and are even hidden from the staff of the clinical trial service provider 12 to render the clinical trial completely “blind”. The same ID will be used for that test subject 44 throughout the clinical trial to match ECG readings and other clinical information, laboratory results, and cardiologists 62 to the test subject 44.

The Subject Polling Module is a software module that forms the central communication between the various modules of the system 10, and specifically establishes integration points between the CTMS 14 and the SMS 22. The SPM 18 also serves as a mechanism to unite the various modules and programs of the system 10 and allow them to communicate using a unified data set. This eliminates data duplication, allows processing of data in real-time and automates the clinical trial process. Also, SPM 18 programs the SMS 22 to follow a set protocol, such as generating ten second samplings of ECG data from test subjects 44. This includes programming the subject IDs into the SMS 22.

The SPM 18 queries the CTMS database and inspects the protocol from the CTMS 14. The SPM 18 automatically imports subject demographics from CTMS 14 and programs the subject demographics into the SMS 22. During the clinical trial, the SPM 18 implements a software-based polling routine or listening routine to spawn listening threads which listen for ECG or other physiological events in the SMS 22 as they occur in real time. This listening routine establishes integration points between the SMS 22 and CTMS 14. When an ECG or other physiological data event occurs, the SPM listening routine acquires the data and automatically transfers the data from the SMS 22 to the CTMS 14 in a data format acceptable to the CTMS 14. The SPM 18 also provides a web-based dashboard where clinical staff can view all the details of the ECG waveforms or other physiological data processed by the SPM 18. The ECG waveforms can be observed in real-time as they are processed allowing the replacement of an ECG if a cardiologist rejects the original and correlates the transfer of the replacement ECG to CTMS 14. The SPM 18 also allows for aspects of the functionality of the SMS 22 to be turned on or off based on the given protocol requirements. The SPM 18 integrates the CAR module's information and data with the CTMS 14 and SMS 22, assuring that the ECG for a given test subject's data is analyzed by the same cardiologist throughout the entire clinical trial.

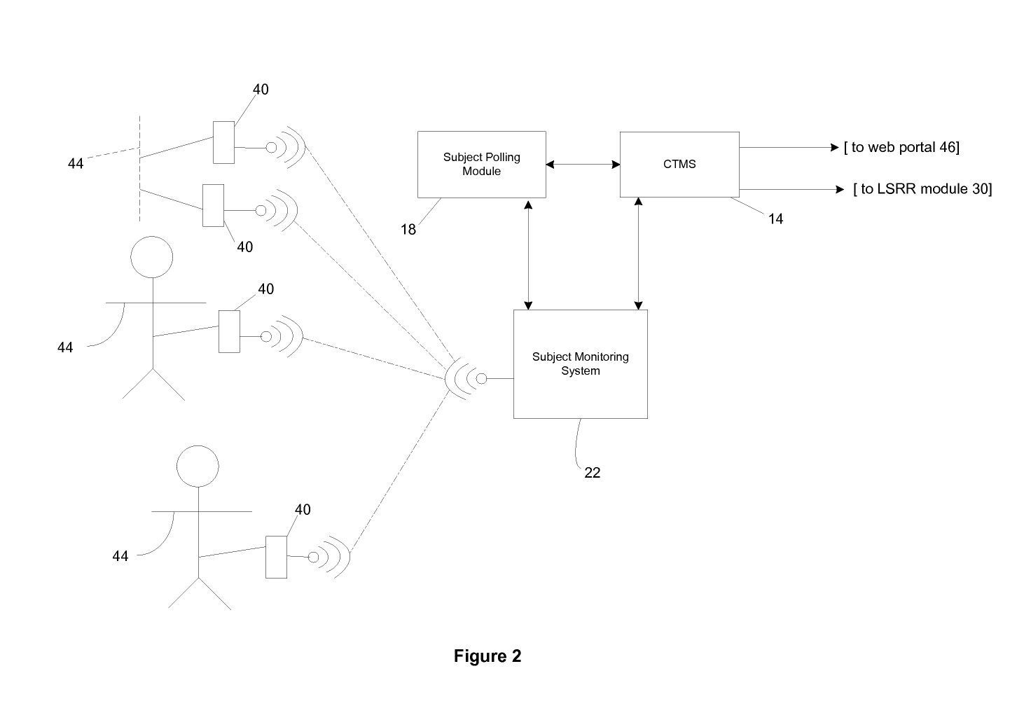

The SMS 22 is a healthcare patient monitoring system for monitoring the clinical parameters of the clinical trial test subjects 44. Monitored parameters can include ECG, heart rate, blood pressure, blood oxygenation, etc. Though not specifically shown, the SMS 22 includes both hardware and software for acquiring the monitored parameters, and for providing some software-based algorithms for interpretation and analysis of the monitored parameters. In the embodiment shown in the drawings (and more specifically in FIG. 2), the SMS 22 is a telemetry-based patient monitoring system that allows the test subjects 44 the freedom to move about the clinical trial environment while still being monitored. A commercially available example of such a system is the Surveyor Telemetry Central System including the Veritas ECG analysis software, both produced and sold by Mortara Instrument, Inc. Test subjects are connected to telemetry transmitters 40 which send monitored parameters wirelessly and continuously to the SMS 22.

The SMS 22 collects ECG data from test subjects 44 continuously. In the preferred embodiment however, the SMS 22 is programmed to transmit the ECG data to the SPM 18 in discrete periods (for example, ten seconds) of the test subject's ECG waveform along with interpretive data relating to the ECG waveform. The timing and duration of these periods are determined by the protocol, and are typically, though not always associated with dosing of the subject 44. The data generated by the SMS 22 includes a text file, such as an XML format file, and an image file, such as a PDF or JEPG format file. The XML file includes a numerical representation of the clinical measurements acquired by the SMS 22, as well as textual data representing the analysis performed and interpretation generated by the SMS 22 on the ECG waveform or other clinical parameters. For example, the XML file includes data relating to the QT interval, QRS complex measurements, RR interval, etc. as well as low level cardiac diagnoses based on those measurements. The PDF file is a visual representation of the ECG waveform. The files are automatically and programmatically parsed into discreet XML and PDF files by the SPM 18, are correlated by the SPM 18 with an ID for that test subject 44, and then transmitted by the SPM 18 to the CTMS 14 for storage associated with the test subject 44.

As stated above, the system 10 also includes Recruitment Module 26. The RM 26 is connected to the subject database 42. The RM 26 is accessed by test subjects 44 over the internet through the web portal 46, through which the test subjects 44 can submit personal information, which is then stored in the subject database 42. The personal information includes individual demographics and health history. Once entered into the subject database 42, the information is reviewed by clinical staff to ensure that the information is complete. The information can be managed and queried by staff managing the clinical trials, and test subjects 44 can be interviewed through telephone screening 52 or in person prior to the clinical trial to ensure the test subjects 44 fit the demographics required of the clinical trial. The telephone screening can be done either by the staff of the clinical trial services provider 12, or by an independent organization (not shown). The RM 26 includes a tracking mechanism allowing statistical analysis on test subjects 44, tracking of the frequency of each test subject's participation in other trials and identification of those trials. Data from the RM 26 is exported in Excel format to the CTMS 14 after it is reviewed by clinical staff. Email notifications may be configured and sent when a test subject 44 enters his/her information, or when an individual test subject 44 in the subject database 42 fits a desired demographic for a clinical trial. The test subject profile is part of the clinical trial protocol, which is generally developed by the sponsor 48 of the clinical trial. In addition, some aspects of the protocol may be dictated by the U.S. Food and Drug Administration (“FDA”).

During evaluation of the test subjects 44 for participation in the clinical trial and during the course of a clinical trial, it may be necessary, depending upon the protocol of the clinical trial, to take physical specimens from the test subjects 44, such as, for example, blood, urine, saliva, etc. for laboratory testing. The scheduling of the laboratory orders are defined by the clinical trial protocol and generated by the CTMS 14 in HL7 format. The LSRR module 30 is a software-based HL7 engine that receives the orders from the CTMS 14 and sends the orders over a secure FTP portal 54 to the laboratory services provider 58. When the laboratory services provider 58 receives the orders, the services provider 58 sends a courier to pick up the specimens and return to the laboratory services provider 58 with the specimens. When the tests on the specimens have been completed, the laboratory services provider 58 posts the results on a server (not shown) and the results are programmatically picked up by the LSRR module 30 through the FTP portal 54, and are transferred to the CTMS 14, where they are stored according to the subject ID. Order and result processing can be disabled to satisfy clinical requirements. Moreover, details of order and result processing can be observed on a dashboard provided by the LSRR module 30. The LSRR module 30 allows one to search for the status or results of individual orders and also group laboratory results into a single reviewable component in the CTMS 14, thereby eliminating the need for data transcription. The CTMS 14 controls the information exchange between the LSRR module 30 and the laboratory services provider 58.

In the course of a clinical trial, review of ECG waveforms acquired during the trial must be done in accordance with federal guidelines. The guidelines include, among other things, a requirement that all ECG waveforms be reviewed by a licensed cardiologist, that no more than a certain number of test subjects 44 can be assigned to a given cardiologist during a clinical trial, that the assignment be totally blind and the initial assignment be random, and that the same cardiologist review ECG waveforms for a given test subject 44. That is, each time a given test subject's ECG waveform is scheduled for cardiologist review, the same cardiologist must conduct the review. The assigning of ECG waveforms to cardiologists 62 (shown conceptually as a group in FIG. 1) in compliance with federal guidelines is done by the CAR module 34 and is achieved through the use of a simple software-based modulus algorithm. Other algorithms can be used for this purpose, and the particular algorithm used is not critical to the invention or operation of the system 10. The guidelines also specify that a certain number of ECG waveforms be re-read, and that of those re-read, a certain number must be re-read by the entire panel of cardiologists (inter-read), and another percentage are simply re-read by the same cardiologist (intra-read). The CAR module 34 automatically schedules re-reads in accordance with the guidelines and provides export (in Excel format) of statistics on the variability of cardiologist ECG interpretations for each cardiologist participating in the cardiologist assignment and review. As with the LSRR module 30, clinical staff can view the details of the cardiologist review through a computer generated dashboard viewable by the clinical staff.

Communication between the cardiologists 62 (working remotely) and the CAR module 34 is performed through the web portal 46. The cardiologists typically utilize a software package to perform their evaluations (also called “over-reads”) such as the E-scribe ECG data management system developed and sold by Mortara Instrument, Inc. Other ECG data management and review systems can of course be substituted. The cardiologist has several choices in submitting an evaluation of the ECG waveforms. First, the cardiologist can accept the waveform and provide a clinical interpretation of the waveform (or alternatively simply confirm the interpretation provided by the SMS 22) through the web portal 46 to the CAR module 34. This interpretation is transmitted from the CAR module 34 to the CTMS 14 for storage associated with the test subject's ID. At the same time, the SPM 18 receives confirmation of the ECG data from the CAR module 34 and correlates the confirmation within the CTMS 14 to “close” the time slot for that ECG data. The time slots (for example, the ten second intervals of ECG data for evaluation, are defined by the protocol programmed into the CTMS 14).

Alternatively, the cardiologist can reject the ECG waveform. In this case, notification is sent through the web portal 46 to the CAR module 34 and by the CAR module 34 to a technician assisting with the clinical trial of the rejected waveform. The technician can review the rejection and the reason for the rejection, and review the ECG waveform data (which is continuously acquired by the SMS 22) to select a different window of ECG waveform data.

Once the revised ECG data is selected, it is re-transmitted to the cardiologist 62 for review and interpretation. In another embodiment, the revised ECG data can be automatically selected by the system 10 automatically choosing either the prior or subsequent ten second period of data. Once approved by the cardiologist, the SPM 18 acquires the new ECG waveform from SMS 22 and replaces the old ECG data stored in the CTMS 14 (at that time slot and for that ID) with the new ECG waveform. The process of automatically, electronically assigning cardiologists to review the data, automatically, electronically presenting the data to the cardiologists, and automatically, electronically acquiring the cardiologists' evaluations means that the cardiologists (while they are working remotely and in parallel with one another) can complete the ECG evaluations in real-time as the clinical trial is being executed.

As shown in FIG. 1, the system 10 is integrated with a video camera network (VCN) 38, and all of the servers and modules of the system 10, including the VCN 38, are synchronized using a common time clock, such as a server connected by satellite to an atomic clock. The VCN 38 includes a plurality of cameras (not shown), each placed in a test subject's room and networked to one another through a common server (not shown). Each camera on the VCN 38 has an IP address that is correlated to the ID for the test subject 44 associated with the room in which the camera is mounted. This allows for acquisition and correlation of patient images with ECG waveform and other patient data for a given time. For example, an ECG waveform might look different if a test subject 44 changes position at the exact time the waveform is acquired. The images acquired by the VCN 38 allow the clinician or clinical trial services provider staff to see what the test subject 44 was doing at the time the ECG waveform or other data was acquired.

In operation, a pharmaceutical company or test sponsor 48 provides the clinical trial service provider with a clinical trial or study protocol. The protocol is entered into the CTMS 14 by the clinical trial service provider staff. In other embodiments, the sponsor 48 can enter the protocol information directly into the CTMS 14 through the web portal 46, the FTP portal 54 or another network entry node (not shown). For example, if the study sponsor 48 wants 10 ECG events per day spaced out 10-15 minutes around test subject dosing and repeated every hour, the information is programmed into the CTMS 14.

The SPM 18 queries the CTMS database and translates the protocol information into an input that can be read by the SMS 22 and the RM 26. The RM 26 effects automatic selection of a group of test subjects 44 based on the study protocols.

The staff of the clinical trial service provider reviews information in the subject database 42 relating to the group, and screens the test subjects 44 telephonically to further refine the test group. Urine and blood samples are collected from the possible test subjects 44. Laboratory tests on the samples are scheduled through the LSRR module 30, which communicates the requests for testing through the secure FTP portal 54 to the laboratory services provider 58. The laboratory services provider 58 schedules a courier, picks up the samples and completes the tests. The laboratory services provider 58 posts the test results in HL7 format to a server, which is then accessed by the LSRR module 30 through the FTP portal 54. The LSRR module 30 then automatically enters the data from the screening and analysis into the CTMS 14. If there are no adverse findings within a test subject's laboratory results, then the test subject 44 may be selected to participate in the study. The clinical staff reviews the results and identifies the final set of the individuals that qualify for the clinical trial. At this time a test subject identifier (“ID”) is generated in the CTMS 14 for each test subject 44, the ID is automatically acquired by the SPM 18, and the ID is programmed into the SMS 22 by the SPM 18.

The SPM 18 performs automatic demographic transcription from the CTMS 14 into the SMS 22. The test subject ID bridges the communication gap between the CTMS 14 and the SMS 22, and the SPM 18 uses the ID to correlate the data between the two systems, I.e., the SMS 22 and the CTMS 14. The automatic nature of the transcription process nullifies any need for manual data entry. Once this process is complete, the SPM 18 generates a protocol file that is used to program the SMS 22. This file is generated by programmatically inspecting the CTMS study data base, and calculating export times for ECG and other physiological data. Once this file is generated and uploaded into the SMS 22, subject ECGs will be exported based on the protocol specified by the sponsor 48.

The subjects 44 are assigned rooms in the clinical trial facility and are connected to the SMS 22, typically the telemetry-based ECG transmitter described previously. The rooms and transmitters are associated with the subject ID. As data is acquired from the patients by the transmitters, the transmitters continuously broadcast the ECG data to the SMS 22. The SMS 22 performs some interpretive algorithms on the data and generates an XML file including the interpretive data, along with the PDF file which is a visual representation of the ECG waveform.

The ECG data files are acquired by the SPM 18 through its listening threads or listening routine and according to the test protocols. The SPM 18 automatically and programmatically parses the XML files from the PDF files, combines the subject ID with the files, and sends all data to the CTMS 14. The association of the data files with the subject ID is achieved using a modulus algorithm in the SPM 18 that correlates the data to the known subject ID. With the ECG waveforms and interpretive data attached to the correct subject IDs, cardiologist review and assessment can begin. The CAR 34 blindly assigns the ECG waveforms to the cardiologists 62 to analyze. In the case that the cardiologist 62 decides that a given sample of ECG data is invalid, inaccurate or is otherwise unacceptable, he/she can reject the ECG data and have the ten second strip replaced automatically. In one embodiment, the selection of alternate ECG data is performed manually by the staff of the clinical trial service provider. The staff performs this function using a dashboard to review the data and select a better data sample to send to the cardiologists 62 for review. In another embodiment, the SPM 18 can automatically select a prior or subsequent period of data for review. As with the original data sample, the new sample is posted to the web portal 46 where it can be viewed and interpreted by the cardiologist 62.

All of the data acquired during the course of the clinical trial is stored in the CTMS 14 and associated with the subject ID. The sponsor 48 and the FDA 50 can be provided with access to the system 10 through web portal 46. Such access would allow either the sponsor 48 or the FDA 50, or both to view the clinical trial process and data acquisition in real time, as the clinical trial is being conducted. Alternatively, access (and therefore automatic reporting of results) could be provided the sponsor 48 or FDA 50 to the results of a clinical trial only when complete. This could be a passive process, whereby the sponsor 48 or FDA 50 are given password protected access to the system 10 to view the results as desired, or an active process, wherein the clinical trial results are sent to the sponsor 48 or FDA 50 by the system 10.

The system 10 provides a useful system and method to automate the implementation of a clinical trial as well as the collection, interpretation, storage and reporting of data from a clinical trial. The system 10 also allows automated, real-time and parallel activities, such as review of ECG and other physiological data, completion of laboratory tests, reporting of laboratory results and storage of all results in a manner associated with a given test subject ID. Various features of the invention are set forth in the following claims.

What is claimed is:

1. A system for automating the implementation of and collection of data during a clinical trial on living subjects, the system comprising:
a computer-based clinical trial management system, programmed to define a clinical trial protocol and to store subject data, including a subject identifier;
an electronic subject monitoring system, for acquiring physiological data from the subject during the clinical trial; and
an electronic subject polling module programmed to acquire the physiological data from the subject monitoring system in accordance with the clinical trial protocol, automatically, electronically associate the physiological data with the appropriate subject identifier and transmit the physiological data to the clinical trial management system for storage.

2. The system as set forth in claim 1, wherein the physiological data acquired by the electronic subject monitoring system is ECG data.

3. The system as set forth in claim 2, wherein the ECG data includes a visual representation of the ECG waveform in an image file format and a numerical representation of the ECG data in a text file format.

4. The system as set forth in claim 3, wherein the subject monitoring system is programmed to include the subject ID in the text file.

5. The system as set forth in claim 2, wherein the ECG data includes a visual representation of the ECG waveform in a PDF file format and a numerical representation of the ECG data in XML file format.

6. The system as set forth in claim 1, wherein the system includes a HL7 engine programmed to perform automatic scheduling of laboratory tests with a laboratory service provider.

7. The system as set forth in claim 6, wherein the HL7 engine automatically receives laboratory results from the laboratory service provider, associates the laboratory results with the subject identifier and stores the laboratory results in the clinical trial management system.

8. The system as set forth in claim 1, wherein the system includes a digital video camera network synchronized with the subject monitoring system.

9. The system as set forth in claim 1, wherein the system includes a digital video camera network and wherein the digital video camera network, the clinical trial management system, the subject monitoring system and the subject polling module are synchronized to a common clock.

10. The system as set forth in claim 1, wherein the system includes a cardiologist assignment and review module programmed to assign cardiologists to review the ECG data.

11. The system as set forth in claim 10, wherein the cardiologist assignment and review module automatically assigns the same cardiologist to the same subject each time analysis from a cardiologist is required.

12. The system as set forth in claim 1, and further comprising a recruitment module accessible through a web portal, and wherein the recruitment module is programmed to electronically receive data relating to subjects and select a group of subjects to participate in the clinical trial based on the protocol.

13. The system as set forth in claim 1, wherein the system and all the server-based applications in the system are synchronized by a single server.

14. The system as set forth in claim 1, wherein the clinical trial management system is programmed to electronically report the results of the clinical trial to a sponsor.

15. A system for automating the implementation of and collection of data during a clinical trial on living subjects, the system comprising:
a computer-based clinical trial management system, programmed to define a clinical trial protocol and to store subject data, including a subject identifier;
an electronic subject monitoring system, for acquiring ECG data from the subject during the clinical trial, the ECG data including an image file representing the ECG waveform, and a text file representing ECG measurements and analysis data;
an electronic subject polling module programmed to acquire the ECG data from the subject monitoring system in accordance with the clinical trial protocol, automatically, electronically associate the physiological data with the appropriate subject identifier and transmit the physiological data to the clinical trial management system for storage; and
a cardiologist assignment and review module, programmed to automatically, electronically assign cardiologists to review the ECG data in accordance with the protocol.

16. The system as set forth in claim 15, wherein the cardiologist assignment and review module automatically assigns the same cardiologist to the same subject each time analysis from a cardiologist is required.

17. The system as set forth in claim 15, wherein the system includes a recruitment module accessible through a web portal, and wherein the recruitment module is programmed to electronically receive data relating to subjects and select a group of subjects to participate in the clinical trial based on the protocol.

18. The system as set forth in claim 15, wherein the system and all the server-based applications in the system are synchronized by a single server.

19. The system as set forth in claim 15, wherein the clinical trial management system is programmed to electronically report the results of the clinical trial to a sponsor.

20. A method of implementing and collecting physiological data during a clinical trial, the method comprising:
developing an electronic database of potential clinical trial subjects;
programming a clinical trial management system with a clinical trial protocol;
automatically, electronically selecting the subjects for the clinical trial from the database based on a comparison of the protocol with the database, and
associating each selected subject with an identifier;
programming a subject monitoring system with the subject identifier and connecting the selected subjects to the subject monitoring system;
acquiring ECG data from the subject monitoring system; and
transmitting the ECG data to the clinical trial management system in accordance with the protocol.
